(19) United States
(12) Patent Application Publication
(10) Pub. No.: US 2026/0266829 A1
(43) Pub. Date: Sep. 10, 2026

(54) C3F Assay

(71) Applicant: Nordic Bioscience A/S, Herlev (DK)

(72) Inventors: Rasmus Sund Petersen, Herlev (DK); Nicholas Willumsen, Gentofte (DK); Morten Asser Karsdal, Kobenhavn Ø (DK)

(73) Assignee: Nordic Bioscience A/S, Herlev (DK)

(21) Appl. No.: 19/167,381

(22) PCT Filed: Mar. 22, 2024
(86) PCT No.: PCT/EP2024/057882

§ 371 Date: Sep. 21, 2025

(30) Foreign Application Priority Data
Mar. 22, 2023 (GB) 2304205.4

(51) Int. Cl. G01N 33/577; C07K 16/18; G01N 33/5752; G01N 33/68

(52) U.S. Cl. CPC G01N 33/577; C07K 16/18; G01N 33/5752; G01N 33/6887; G01N 2800/105; G01N 2800/7028

(57) ABSTRACT

Disclosed herein are methods of immunoassay for detecting a fibroblast activation protein (FAP) generated fragment of type III collagen in a patient sample, and the use thereof for detecting and/or monitoring cancer or arthritis or a particular level of severity thereof in a patient. Also described are monoclonal antibodies and assay kits for use in said methods of immunoassay.

### FIELD OF THE INVENTION

The present invention relates to methods of immunoassay for detecting a fibroblast activation protein (FAP) generated fragment of type III collagen in a patient sample, and the use thereof for detecting and/or monitoring cancer or arthritis or a particular level of severity thereof in a patient. The present invention also relates to monoclonal antibodies and assay kits for use in said methods of immunoassay.

### BACKGROUND

With an estimated more than 230,000 new lung cancer cases and more than 130,000 deaths due to lung cancer in 2022 in the United States alone, lung cancer remains one of the leading causes of cancer related death^{[1]}.

It is well established that the tumor microenvironment (TME) is important for tumor progression and patient survival, and as such there is an increasing interest in the TME for investigation and treatment of various cancers^{[2-5]}. The TME includes the extracellular matrix (ECM), tumor cells, immune cells, and stromal cellsl^{[6]}.

Cancer associated fibroblasts (CAFs) are a major component of the tumor stroma^{[6,7]}. CAFs are involved in cancer progression, immunosuppression, and ECM remodeling^{[10]}. CAFs affect ECM remodeling by excess synthesis of collagens and other ECM components and by secreting metalloproteinases (MMPs)^{[}10,11].

Fibroblast activation protein (FAP) is a type II transmembrane serine protease that is primarily expressed in the stroma and almost exclusively under pathological conditions including arthritis, fibrosis and cancer, leading it to be one of the primary biological markers of CAFs^{[3,12,13]}.

Several studies have investigated the diagnostic and prognostic value of FAP^{[2,14-17]}. While the results of these studies have been somewhat inconclusive as regards the general prognostic value of FAP, FAP has been shown to be a potential prognostic marker for outcome in lung, stomach, and colon cancer^{[16-18]}. FAP has also been shown to be involved in ECM remodeling and fibrogenesis^{[19]}.

Excessive ECM degradation, e.g. from MMP mediated cleavage during tumor progression, results in protein fragments that are released into circulation. These fragments can be assessed non-invasively as a liquid biopsy and quantified as a disease biomarker^{[20,21]}. As FAP have been shown to cleave ECM components including type I and III collagen, it can be expected that fragments from these cleavages would appear in circulationE^{[12,22]}.

FAP belongs to the dipeptidyl peptidase 4 (DPP4) protein family and like the other enzymes in the DPP4 family, FAP has dipeptidyl peptidase activity. In addition, FAP also has endopeptidase activity, which separates FAP from other DPP4 proteins and leads to potentially unique cleavage targets^{[3,13]}.

Zhang et al. used terminal amine isotopic labelling of substrates (TAILS) for a mouse embryonic fibroblast to identify natural substrate of human FAP. Using this degradomic method, Zhang et al. identified multiple potential FAP cleavage site in various ECM-associated proteins, including ¹⁰⁶⁹AGPSGAPGPA¹⁰⁷⁸ (SEQ ID NO: 20) on mouse type III collagen^{[22]}.

### SUMMARY OF THE INVENTION

The present inventors have now developed an enzyme-linked immunosorbent assay (ELISA) targeting fragments of the alpha-1 chain of human type III collagen generated by cleavage of the alpha-1 chain between amino acid residues P¹⁰⁶⁹ and A¹⁰⁷⁰ by fibroblast activation protein (FAP), which fragments can serve as a biomarker of FAP activity in the ECM. The inventors have furthermore measured the levels of said biomarker in a lung cancer cohort and compared the levels in patients with non-small cell lung (including adenocarcinoma and squamous cell carcinoma) to healthy subjects. The inventors have also measured the levels of said biomarker in patients with spondyloarthritis. The inventors have demonstrated that the levels of said biomarker were elevated in the patients with cancer and elevated in the patients with arthritis.

Accordingly, in a first aspect the present invention provides a method of immunoassay, said method comprising contacting a patient sample with a monoclonal antibody that specifically binds to a fibroblast activation protein (FAP) generated neo-epitope of a FAP-generated fragment of the alpha-1 chain of human type III collagen, and detecting and determining the amount of binding between said monoclonal antibody and peptides in the sample, wherein the FAP-generated neo-epitope consists of an N-terminus or C-terminus sequence of the FAP-generated fragment at an end of the FAP-generated fragment that has been cleaved by FAP.

In a preferred embodiment, the method of immunoassay comprises;
i) contacting a patient sample with a monoclonal antibody that specifically binds to the N-terminus amino acid sequence AGPAGAPGPA (SEQ ID NO: 1) (said sequence also being referred to herein as the “target sequence” and/or “C3F”); and
ii) detecting and determining the amount of binding between said monoclonal antibody and peptides in the sample.

In a preferred embodiment, the method of immunoassay is a method of immunoassay for detecting and/or monitoring a cancer or a particular level of severity thereof in a patient, the method further comprising correlating said amount of binding with values associated with normal healthy subjects and/or values associated with known disease severity and/or values obtained from said patient at a previous time point and/or with a predetermined cut-off value.

In a preferred embodiment, the cancer is a lung cancer. In a preferred embodiment, the lung cancer is a non-small cell lung cancer (NSCLC).

In another preferred embodiment, the method of immunoassay is a method of immunoassay for detecting and/or monitoring an arthritis or a particular level of severity thereof in a patient, the method further comprising correlating said amount of binding with values associated with normal healthy subjects and/or values associated with known disease severity and/or values obtained from said patient at a previous time point and/or with a predetermined cut-off value.

In a preferred embodiment, the arthritis is is spondyloarthritis.

In a preferred embodiment, the patient sample is a human biofluid sample. Preferably the sample is a blood-based sample, such as blood (whole blood), plasma or serum.

In a preferred embodiment, the monoclonal antibody does not specifically bind to a peptide having the N-terminus amino acid sequence PAGPAGAPGPA (SEQ ID NO: 2) (i.e. an elongated version of the target sequence extended at its N-terminus by the addition of a proline residue). Preferably, the ratio of the affinity of said antibody for said target sequence to the affinity of said antibody for the elongated version of the target sequence is at least 10 to 1, and more preferably is at least 20 to 1, at least 30 to 1, at least 40 to 1, at least 50 to 1 or at least 100 to 1.

In a preferred embodiment, the monoclonal antibody not specifically bind to a peptide having the N-terminus amino acid sequence GPAGAPGPA (SEQ ID NO: 3) (i.e. a truncated version of the target sequence truncated at its N-terminus by the removal of the first alanine residue). Preferably, the ratio of the affinity of said antibody for said target sequence to the affinity of said antibody for the truncated version of the target sequence is at least 10 to 1, and more preferably is at least 20 to 1, at least 30 to 1, at least 40 to 1, at least 50 to 1 or at least 100 to 1.

In a preferred embodiment, the monoclonal antibody is raised against a synthetic peptide having the N-terminus amino acid sequence AGPAGAPGPA (SEQ ID NO: 1). For example, the monoclonal antibodies may be raised by: (a) immunizing a rodent (or other suitable mammal) with a synthetic peptide comprising the N-terminus sequence AGPAGAPGPA (SEQ ID NO: 1), which peptide may optionally be linked at its C-terminus to an immunogenic carrier protein (such as keyhole limpet hemocyanin (“KLH”)); (b) isolating and cloning a single antibody producing cell; and (c) assaying the resulting monoclonal antibodies to ensure that they have the desired specificity. An exemplary protocol of the development, production and characterization of suitable monoclonal antibodies is described in the Examples section, infra.

In preferred embodiments the immunoassay is a competition assay or a sandwich assay. The immunoassay may, for example, be a radio-immunoassay or an enzyme-linked immunosorbent assay (ELISA). Such assays are techniques known to the person skilled in the art.

As used herein the term “N-terminus” refers to an N-terminal peptide sequence at the extremity of a polypeptide, i.e. at the N-terminal end of the polypeptide, and is not to be construed as meaning in the general direction thereof. As used herein the term “C-terminus” refers to a C-terminal peptide sequence at the extremity of a polypeptide, i.e. at the C-terminal end of the polypeptide, and is not to be construed as meaning in the general direction thereof.

As used herein, the terms “peptide” and “polypeptide” are used synonymously.

As used herein the term “monoclonal antibody” refers to both whole antibodies and to fragments thereof that retain the binding specificity of the whole antibody, such as for example a Fab fragment, F(ab′)2 fragment, single chain Fv fragment, or other such fragments known to those skilled in the art. As is well known, whole antibodies typically have a “Y-shaped” structure of two identical pairs of polypeptide chains, each pair made up of one “light” and one “heavy” chain. The N-terminal regions of each light chain and heavy chain contain the variable region, while the C-terminal portions of each of the heavy and light chains make up the constant region. The variable region comprises three complementarity determining regions (CDRs), which are primarily responsible for antigen recognition. The constant region allows the antibody to recruit cells and molecules of the immune system. Antibody fragments retaining binding specificity comprise at least the CDRs and sufficient parts of the rest of the variable region to retain said binding specificity.

In the present invention, a monoclonal antibody comprising any constant region known in the art can be used. In the case of mouse antibodies and human antibodies, the constant light chains are classified as either kappa or lambda light chains.

Heavy constant chains are classified as mu, delta, gamma, alpha, or epsilon, and define the antibody's isotype as IgM, IgD, IgG, IgA, and IgE, respectively. The IgG isotype has several subclasses, including, but not limited to IgG1, IgG2, IgG3, and IgG4 in the case of humans and IgG1, IgG2a, IgG2b, IgG2c and IgG3 in the case of mice. The monoclonal antibody may preferably be of the IgG isotype, including any one of the IgG subclasses.

The CDR of an antibody can be determined using methods known in the art such as that described by Kabat et al. Antibodies can be generated from B cell clones. The isotype of the antibody can be determined by ELISA specific for IgM, IgG or IgA isotype, or subclass. The amino acid sequence of the antibodies generated can be determined using standard techniques. For example, RNA can be isolated from the cells, and used to generate cDNA by reverse transcription. The cDNA is then subjected to PCR using primers which amplify the heavy and light chains of the antibody. For example, primers specific for the leader sequence for all VH (variable heavy chain) sequences can be used together with primers that bind to a sequence located in the constant region of the isotype which has been previously determined. The light chain can be amplified using primers which bind to the 3′ end of the Kappa or Lamda chain together with primers which anneal to the V kappa or V lambda leader sequence. The full length heavy and light chains can be generated and sequenced.

In certain exemplary embodiments, the monoclonal antibody may preferably comprise one or more complementarity-determining regions (CDRs) selected from:

CDR-L1:
(SEQ ID NO: 4)
KSSQSLVYSDGKTYMN
CDR-L2:
(SEQ ID NO: 5)
LVSKLDS
CDR-L3:
(SEQ ID NO: 6)
WQGTHSPLT
CDR-H1:
(SEQ ID NO: 7)
SYWMN
CDR-H2:
(SEQ ID NO: 8)
QIYPGTGDTNYNGKFKG
CDR-H3:
(SEQ ID NO: 9)
ITTAYYFDY

Preferably the monoclonal antibody comprises at least 2,3,4,5 or 6 of the above listed CDR sequences.

Preferably the monoclonal antibody has a light chain variable region comprising the CDR sequences:

CDR-L1:
(SEQ ID NO: 4)
KSSQSLVYSDGKTYMN
CDR-L2:
(SEQ ID NO: 5)
LVSKLDS
and
CDR-L3:
(SEQ ID NO: 6)
WQGTHSPLT.

Preferably the monoclonal antibody has a light chain that comprises framework sequences between the CDRs, wherein said framework sequences are substantially identical or substantially similar to the framework sequences between the CDRs in the light chain sequence below (in which the CDRs are shown in bold and underlined, and the framework sequences are shown in italics)

(SEQ ID NO: 10)
KSSQSLVYSDGKTYMNWLLQRPGQSPKRLVYLVSKLDSGVPDRFTGSGS
GTDFTLKISRVEAEDLGVYYCWQGTHSPLT

Preferably the monoclonal antibody has a heavy chain variable region comprising the CDR sequences:

CDR-H1:
(SEQ ID NO: 7)
SYWMN
CDR-H2:
(SEQ ID NO: 8)
QIYPGTGDTNYNGKFKG
and
CDR-H3:
(SEQ ID NO: 9)
ITTAYYFDY.

Preferably the monoclonal antibody has a heavy chain that comprises framework sequences between the CDRs, wherein said framework sequences are substantially identical or substantially similar to the framework sequences between the CDRs in the heavy chain sequence below (in which the CDRs are shown in bold and underlined, and the framework sequences are shown in italics)

(SEQ ID NO: 11)
SYWMNWVKQSPGQGLEWIGQIYPGTGDTNYNGKFKGKVTLTTDKSSSTS
YMQLSSLTSEDSAVYFCVLITTAYYFDY

Preferably, the monoclonal antibody comprises the light chain variable region sequence:

(SEQ ID NO: 12)
DVVMTQTPRTLSVIIGQPASISCKSSQSLVYSDGKTYMNWLLQRPGQSP
KRLVYLVSKLDSGVPDRFTGSGSGTDFTLKISRVEAEDLGVYYCWQGTH
SPLTFGAGTKLELK
(CDRs bold and underlined; Framework sequences in
italics)

and/or the heavy chain variable region sequence:

(SEQ ID NO: 13)
QVQVQQSGAELVRPGSSVKISCKASGYVFSSYWMNWVKQSPGQGLEWIG
QIYPGTGDTNYNGKFKGKVTLTTDKSSSTSYMQLSSLTSEDSAVYFCVL
ITTAYYFDYWGQGTTLTVSS
(CDRs bold and underlined; Framework sequences in
italics)

As used herein, the framework amino acid sequences between the CDRs of an antibody are “substantially identical” or “substantially similar” to the framework amino acid sequences between the CDRs of another antibody if they have at least 70%, 80%, 90% or at least 95% similarity or identity. The similar or identical amino acids may be contiguous or non-contiguous. The framework sequences may contain one or more amino acid substitutions, insertions and/or deletions. Amino acid substitutions may be conservative, by which it is meant the substituted amino acid has similar chemical properties to the original amino acid. A skilled person would understand which amino acids share similar chemical properties. For example, the following groups of amino acids share similar chemical properties such as size, charge and polarity: Group 1 Ala, Ser, Thr, Pro, Gly; Group 2 Asp, Asn, Glu, Gln; Group 3 His, Arg, Lys; Group 4 Met, Leu, Ile, Val, Cys; Group 5 Phe Thy Trp.

A program such as the CLUSTAL program to can be used to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of analysis are contemplated in the present invention. Identity or similarity is preferably calculated over the entire length of the framework sequences.

As used herein the term “amount of binding” refers to the quantification of binding between the antibody and peptides in the patient sample. Said quantification may for example be determined by comparing the measured values of binding in the patient sample against a calibration curve produced using measured values of binding in standard samples containing known concentrations of a peptide to which the antibody specifically binds, in order to determine the quantity of peptide to which the antibody specifically binds in the patient sample. Any suitable analytical method can be used for measuring the amount of binding. For example, an ELISA method can be used in which spectrophotometric analysis is used to measure the amount of binding both in the patient samples and when producing the calibration curve.

As used herein the term “predetermined cut-off value” means an amount of binding that is determined statistically to be indicative of a high likelihood of a disease (e.g. a cancer or an arthritis) or a particular severity thereof in a patient, in that a measured value of the target peptide in a patient sample that is at or above the statistical cut-off value corresponds to at least a 70% probability, preferably at least an 75% probability, more preferably at least an 80% probability, more preferably at least an 85% probability, more preferably at least a 90% probability, and most preferably at least a 95% probability of the presence of said disease or particular severity thereof.

As used herein, the term “values associated with normal healthy subjects” means standardised quantities of binding determined by the method described supra for samples from subjects considered to be healthy, i.e. without disease (i.e. without cancer or without arthritis); and the term “values associated with known disease severity” means standardised quantities of binding determined by the method described supra for samples from patients known to have disease (i.e. a cancer or an arthritis) of a known severity.

In a second aspect, the present invention provides a method of a cancer in a patient in need thereof, the method comprising:
(a) carrying out a method of immunoassay for detecting a cancer or a particular level of severity thereof in accordance with the first aspect of the present invention on a sample from a patient; and
(b) administering to the patient a therapy for the treatment of the cancer if it is determined in step (a) that the patient has said cancer or said particular level of severity thereof.

The therapy may be any therapy suitable for treating the cancer in question. The therapy may for example comprise or consist of one or more surgeries, one or more radiation therapies, one or more medicaments (such as for example one or more chemotherapies, one or more immunotherapies and/or one or more hormonal therapies), or combinations thereof. Medicaments may be formulated to topical or systemic administration. Topical medicaments may for example be formulated as creams, foams, gels, lotions, or ointments for administration. Systemic medicaments may for example be formulated for enteral or parenteral administration. Surgeries may be curative surgeries, preventative surgeries, debulking surgeries, palliative surgeries and/or restorative surgeries.

For example, where the cancer is lung cancer (such as for example NSCLC), suitable therapies may for example comprise one or more of: surgery, such as performing a lung resection such as for example a lobectomy, a sublobar excision (wedge resection) or removal of a whole lung (pneumonectomy); radiotherapy, examples of which include but are not limited to radiotherapy given together with chemotherapy, post-operative radiotherapy, brachytherapy (localized radiotherapy), prophylactic cranial irradiation, stereotactic radiation and palliative radiotherapy; chemotherapy using for example one more agents such as cisplatin, carboplatin, etoposide, gemcitabine, paclitaxel, docetaxel, vinorelbine, topotecan, irinotecan and pemetrexed; epidermal growth factor receptor (EGFR) inhibitor drugs such as erlotinib, gefitinib, afatinib, dacomitinib or osimertinib; targeted therapies using one or more drugs such as for example crizotinib and immunotherapy, using one or more monoclonal antibodies such as for example anti PD-Li monoclonal antibodies such as atezolizumab, nivolumab or pembrolizumab, monoclonal antibodies targeting cytotoxic T-lymphocyte-associated protein 4 (CTLA-4) such as ipilimumab, and/or monoclonal antibodies that target vascular endothelial growth factor such as bevacizumab, necitumumab, mobocertinib or cetuximab. In a third aspect, the present invention provides a method of an arthritis in a patient in need thereof, the method comprising:
(a) carrying out a method of immunoassay for detecting an arthritis a particular level of severity thereof in accordance with the first aspect of the present invention on a sample from a patient; and
(b) administering to the patient a therapy for the treatment of the arthritis if it is determined in step (a) that the patient has said arthritis or said particular level of severity thereof.

The therapy may be any therapy suitable for treating the arthritis in question. The therapy may for example comprise or consist of one or more surgeries, one or more physical therapies, one or more medicaments, or combinations thereof. Medicaments may be formulated to topical or systemic administration. Topical medicaments may for example be formulated as creams, foams, gels, lotions, or ointments for administration. Systemic medicaments may for example be formulated for enteral or parenteral administration.

For example, where the arthritis is spondyloarthritis suitable therapies may for example comprise one or more immunosuppressive drugs, such as for example one or more tumor necrosis factor (TNF) inhibitors.

In a fourth aspect, the present invention provides a monoclonal antibody that specifically binds to the N-terminus amino acid sequence AGPAGAPGPA (SEQ ID NO: 1).

The antibody according to the fourth aspect of the invention is, in particular, suitable for use in carrying out the methods of immunoassay according to the first aspect of the invention. Preferred embodiments and features of the antibody according to the third aspect will therefore be apparent from the above discussion of the preferred embodiments of the methods according to the first aspect.

In a fifth aspect, the present invention provides an immunoassay kit comprising a monoclonal antibody in accordance with the fourth aspect of the present invention, and at least one of:
a streptavidin coated well plate
a biotinylated peptide AGPAGAPGPA-L-Biotin (SEQ ID NO: 14) wherein L is an optional linker
a secondary antibody for use in a sandwich immunoassay
a calibrator protein comprising the N-terminus amino acid sequence AGPAGAPGPA (SEQ ID NO: 1)
an antibody biotinylation kit
an antibody HRP labelling kit
an antibody radiolabelling kit

The immunoassay kit according to the fifth aspect of the invention is, in particular, suitable for use in carrying out the method of immunoassay according to the first aspect of the invention. Further preferred embodiments and features of the immunoassay kit according to the fifth aspect will therefore be apparent from the above discussion of the preferred embodiments of the methods according to the first aspect.

### FIGURES

FIG. 1: Specificity of the C3F ELISA. (A) Specificity of the C3F antibody and assay towards the 10aa selection peptide (AGPAGAPGPA (SEQ ID NO: 1)), 30aa selection peptide (AGPAGAPGPAGSRGAPGPQGPRGDKGETGE (SEQ ID NO: 19)) truncated peptide (GPAGAPGPA (SEQ ID NO: 3)), and elongated peptide (PAGPAGAPGPA (SEQ ID NO: 2)). (B) Specificity of the C3F ELISA towards FAP cleaved type III collagen, as shown by C3F measurements of collagen III solution after 24 h of incubation with FAP.

FIG. 2: Specificity of the C3F ELISA towards FAP cleaved type III collagen, as shown by (A) C3F measurements and (B) C3M measurements of collagen III solution after 24 h of incubation with FAP or MMP9.

FIG. 3: C3F and C3M levels in serum of patients with lung cancer. Quantification of C3F (A and C) and C3M (B and D) in serum from healthy subjects (n=26) and patients with lung cancer (n=40) combined (A-B) and divided into patients with adenocarcinoma (n=26) or squamous cell carcinoma (n=14) (C-D). Biomarker levels between the groups are compared using Mann-Whitney test (A-B) and Kruskal-Wallis test followed by Dunn's multiple comparisons test (C-D). ns indicates p>0.05, * indicates 0.01<p<0.05, *** indicates 0.001<p<0.0001, and **** indicates p<0.0001.

FIG. 4: Diagnostic accuracy of the C3F and C3M assays. AUROC for separation of healthy and patients with Adenocarcinoma (A and C) and of patients with Adenocarcinoma and Squamous cell carcinoma (B and D) based on serum levels of C3F (A-B) or C3M (C-D).

FIG. 5: Correlation between C3F and C3M. C3F and C3M values from patients with Adenocarcinoma (triangles) or Squamous cell carcinoma (circles). Correlation between C3F and C3M was tested with Spearman correlation. Spearman's r and p-value are shown on the figure.

FIG. 6: C3F levels in serum of patients with lung cancer. (A) Quantification of C3F in serum from healthy subjects (n=42) and patients with NSCLC (n=109). Biomarker levels between the groups are compared using Mann-Whitney test. (B) AUROC for separation of healthy subjects and patients with NSCLC based on serum levels of C3F. ns indicates p>0.05, * indicates 0.01<p<0.05, *** indicates 0.001<p<0.0001, and **** indicates p<0.0001.

FIG. 7: Correlation between C3F and C3M. C3F and C3M values from patients with NSCLC (n=32). Correlation between C3F and C3M was tested with Spearman correlation. Spearman's r and p-value are shown on the figure.

FIG. 8: C3F levels in serum of patients with spondyloarthritis. (A) Quantification of C3F (in serum from healthy subjects (n=19) and patients with Spondyloarthritis (SpA) (n=17). Biomarker levels between the groups are compared using Mann-Whitney test. (B) AUROC for separation of healthy subjects and patients with SpA based on serum levels of C3F. ns indicates p>0.05, * indicates 0.01<p<0.05, *** indicates 0.001<p<0.0001, and **** indicates p<0.0001.

### EXAMPLES

The presently disclosed embodiments are described in the following Examples, which are set forth to aid in the understanding of the disclosure, and should not be construed to limit in any way the scope of the disclosure as defined in the claims which follow thereafter. The following examples are put forth so as to provide those of ordinary skill in the art with a complete disclosure and description of how to make and use the described embodiments, and are not intended to limit the scope of the present disclosure nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Materials and Methods

### Monoclonal Antibody Development, Production, and Characterization

A monoclonal antibody (mAb), specific for the N-terminus amino acid sequence ¹⁰⁷⁰AGPAGAPGPA¹⁰⁷⁹ (SEQ ID NO: 1) (corresponding to the N-terminus of the C-terminal fragment of the alpha-1 chain of type III collagen generated on cleavage of the alpha-1 chain of type III collagen (UniprotKB: P02461) between amino acid residues P¹⁰⁶⁹ and A¹⁰⁷⁰ by fibroblast activation protein (FAP)), was raised using the following procedure.

A 10 amino-acid peptide AGPAGAPGPA (SEQ ID NO: 1) was purchased from Genscript (Piscataway, NJ, USA) for use as the target peptide. An immunogenic peptide (AGPAGAPGPA-GGC-“KLH” (SEQ ID NO: 15)) was generated by covalently cross-linking the target peptide to Keyhole Limpet Hemocyanin (“KLH”) carrier protein using sulfosuccinimidyl 4-(N-maleimidomethyl) cyclohexane-1-carboxylate, SMCC (Thermo Scientific, Waltham, MA, USA, cat.no. 22322). Glycine and cysteine residues were added at the C-terminal end of the target peptide as a linker to ensure correct linking of the carrier protein to the target peptide.

Five female Balb/C mice of 6-7 weeks of age were immunized subcutaneously with 200 μl emulsified antigen and 100 μg immunogenic peptide using Sigma adjuvant system (Sigma). The immunizations were repeated every second week until stable serum antibody titer levels were reached. The mouse with the highest serum titer was chosen for fusion and rested for a month. Then, the selected mouse was boosted intravenously with 50 μg immunogenic peptide in 100 μl 0.9% NaCl solution 3 days before isolation of the spleen for cell fusion. The mouse spleen cells were fused with SP2/0 myeloma cells to produce hybridoma cells as described by Gefter et al., Hybridoma cells were plated in individual wells into 96-well microtiter plates for further growth using the limiting dilution method to promote monoclonal growth. An indirect ELISA performed on streptavidin-coated 96-well microtiter plates was used to screen supernatants from hybridoma clones for reactivity to identify the best mAb-producing clones. The assay employed a biotin-labeled screening peptide (AGPAGAPGPA-K-Biotin (SEQ ID NO: 16)) and tested antibody specificity using selection (AGPAGAPGPA (SEQ ID NO: 1)) and deselection (elongated (PAGPAGAPGPA (SEQ ID NO: 2)) and truncated (GPAGAPGPA (SEQ ID NO: 3))) peptides. The best clone was selected based on specificity to the selection peptide and no reaction to the deselection peptides. Supernatant was harvested and mAb was purified using HiTrap affinity columns (GE Healthcare Life Science, Little Chalfront, Buckinghamshire, UK).

Finally, antibody isotype was determined using the SBA Clonetyping System-HRP kit (SouthernBiotech, Birmingham, AL, USA), and the sequence and CDRs of the monoclonal antibody were determined. All procedures were performed according to the manufacturers' instructions.

The sequence of the chains of the monoclonal antibody are as follows (CDRs underlined and in bold; N-terminus signal peptide and C-terminus Constant region in italics):

Heavy Chain Sequence (Mouse IgG2b isotype)
(SEQ ID NO: 17)
MEWPCIFLFLLSVTEGVHSQVQVQQSGAELVRPGSSVKISCKASGYVFS
SYWMNWVKQSPGQGLEWIGQIYPGTGDTNYNGKFKGKVTLTTDKSSSTS
YMQLSSLTSEDSAVYFCVLITTAYYFDYWGQGTTLTVSSAKTTPPSVYP
LAPGCGDTTGSSVTLGCLVKGYFPESVTVTWNSGSLSSSVHTFPALLQS
GLYTMSSSVTVPSSTWPSQTVTCSVAHPASSTTVDKKLEPSGPISTINP
CPPCKECHKCPAPNLEGGPSVFIFPPNIKDVLMISLTPKVTCVVVDVSE
DDPDVQISWFVNNVEVHTAQTQTHREDYNSTIRVVSTLPIQHQDWMSGK
EFKCKVNNKDLPSPIERTISKIKGLVRAPQVYILPPPAEQLSRKDVSLT
CLVVGFNPGDISVEWTSNGHTEENYKDTAPVLDSDGSYFIYSKLNMKTS
KWEKTDSFSCNVRHEGLKNYYLKKTISRSPGK
Light Chain Sequence (Mouse Kappa Isotype)
(SEQ ID NO: 18)
MSPAQFLLLSVFWIREIKGDVVMTQTPRTLSVIIGQPASISCKSSQSLV
YSDGKTYMNWLLQRPGQSPKRLVYLVSKLDSGVPDRFTGSGSGTDFTLK
ISRVEAEDLGVYYCWQGTHSPLTFGAGTKLELKRADAAPTVSIFPPSSE
QLTSGGASVVCFLNNFYPKDINVKWKIDGSERQNGVLNSWTDQDSKDST
YSMSSTLTLTKDEYERHNSYTCEATHKTSTSPIVKSFNRNEC

### C3F ELISA Protocol

The development of a competitive ELISA (also referred to herein as the “C3F assay”) employing the mAb described above and targeting the N-terminus amino acid sequence AGPAGAPGPA (SEQ ID NO: 1) (also referred to herein as “C3F”) included several preliminary optimizing experiments where various parameters, including assay buffers, incubation time and temperature, and concentrations of antibodies and peptides were optimized.

The final C3F assay procedure was as follows: a 96-well streptavidin-coated ELISA plate was coated with 100 μL/well of 3.5 ng/mL of biotinylated peptide (AGPAGAPGPA-K-Biotin (SEQ ID NO: 16)) dissolved in assay buffer (25 mM PBS, 1% BSA (w/v), 0.018% bronidox (v/v), 0.1% Tween-20 (w/v), 2 g/L NaCl, pH 7.4) incubated with shaking (300 rpm) for 30 minutes at 20° C. in darkness. After washing 5 times with washing buffer (25 mM Tris, 50 mM NaCl, pH 7.2), 20 μL/well of pre-diluted sample (1:4 in initial versions of the assay procedure, but in the final version of the assay procedure pre-diluted to 1:2 instead) was added in duplicates followed by 100 μL/well of 12 ng/mL monoclonal antibody in assay buffer and incubated with shaking (300 rpm) for 20 hours at 4° C. in darkness. After another washing cycle (5 times with washing buffer) 100 μL/well of goat anti-mouse IgG Secondary antibody, HRP (ThermoFisher cat #31437) diluted in assay buffer to a final dilution of 1:5000 was added followed by 1 hour of incubation with shaking (300 rpm) for 1 hour at 20° C. in darkness. After a final washing cycle, 100 μL/well of 3,3′,5,5′-Tetramethylbenzidine (TMB) was added and incubated with shaking (300 rpm) for 15 minutes at 20° C. in darkness. The reaction was stopped by adding 100 μL/well of 1% H₂SO₄. Absorbance was measured at 450 nm with 650 nm as reference. The standard curve was generated by adding 20 μL/well of 800 nM of a 30aa long standard peptide (AGPAGAPGPAGSRGAPGPQGPRGDKGETGE (SEQ ID NO: 19)) serially diluted twofold and a four-parameter logistic (4PL) model was used to fit a curve.

### Specificity of the C3F ELISA

The specificity of the antibody towards the 10aa long selection peptide (¹⁰⁷⁰AGPAGAPGPA¹⁰⁷⁹ (SEQ ID NO: 1)) was assessed by comparing binding to the selection peptide with binding to elongated (¹⁰⁶⁹PAGPAGAPGPA¹⁰⁷⁹ (SEQ ID NO: 2)) and truncated (¹⁰⁷¹GPAGAPGPA¹⁰⁷⁹ (SEQ ID NO: 3)) versions of the peptide. Furthermore, recombinant type III collagen (Abcam cat #ab7535) was incubated with FAP (Bio-techne, cat #3715-SE) or 4-Aminophenylmercuric acetate (Sigma-Aldrich cat #A9563) activated MMP-9 (Bio-techne cat #911-MP) for 24 hours followed by measurement of C3F and C3M (an established ELISA for measuring MMP-9 cleaved type III collagen (Nordic Bioscience cat #1200AG01)) to show the specificity towards FAP cleaved type III collagen.

### Patient Samples—First Cohort

C3F and C3M levels were measured in a lung cancer cohort with serum from 26 healthy subjects, 26 patients with adenocarcinoma and 14 patients with squamous cell carcinoma. Cancer serum samples were purchased from Proteogenex (Los Angeles, CA, USA) and the healthy controls were obtained from BioIVT (Westbury, NY, USA). Details of the cohort are given in Table 1.

TABLE 1
Demographics of the cohort

| | Adenocarcinoma | | Squamous cell | | | |
|---|---|---|---|---|---|---|
| | lung Cancer | | carcinoma lung cancer | | Healthy | |
| Characteristic | (n = 26) | | (n = 14) | | (n = 26) | |
| Stages, n (%) | | | | | | |
| III | 3 | (12) | 2 | (14) | n/a | |
| IV | 23 | (88) | 12 | (86) | n/a | |
| Age, Mean (SD) | 61 | (8) | 59 | (6) | 40 | (12) |
| Sex, n (%) | | | | | | |
| Male | 19 | (73) | 12 | (86) | 11 | (42) |
| Female | 7 | (27) | 2 | (14) | 15 | (58) |

### Patient Samples—Second Cohort

C3F levels were measured in a second lung cancer cohort with serum from 42 healthy subjects and 109 patients with NSCLC. C3M was measured in serum from 32 of these patients with NSCLC. Cancer serum samples were purchased from Proteogenex (Los Angeles, CA, USA) and the healthy controls were obtained from BioIVT (Westbury, NY, USA). Details of the cohort are given in Table 2.

TABLE 2
Demographics of the cohort

| | | |
|---|---|---|
| | Non-small | |
| | cell lung Cancer | Healthy |
| Characteristic | (n = 109) | (n = 42) |

| Subtype, n (%) | | | | |
|---|---|---|---|---|
| Adenocarcinoma | 66 | (61) | n/a | |
| Squamous cell | 43 | (39) | n/a | |
| carcinoma | | | | |
| Stages, n (%) | | | | |
| III | 40 | (37) | n/a | |
| IV | 69 | (63) | n/a | |
| Age, Mean | 62 | (7) | 54 | (9) |
| (SD) | | | | |
| Sex, n (%) | | | | |
| Male | 77 | (71) | 30 | (71) |
| Female | 32 | (29) | 12 | (29) |

### Patient Samples—Third Cohort

C3F levels were also measured in a spondyloarthritis cohort with serum from 19 healthy subjects and 17 patients with spondyloarthritis. Details of the cohort are given in Table 3.

TABLE 3
Demographics of the cohort

| | | |
|---|---|---|
| | Spondyloarthritis | Healthy |
| Characteristic | (n = 17) | (n = 19) |

| | | | | |
|---|---|---|---|---|
| Age, Mean | 49 | (X) | 51 | (X) |
| (SD) | | | | |
| Sex, n (%) | | | | |
| Male | 10 | (59) | 11 | (58) |
| Female | 7 | (41) | 8 | (42) |

### Statistics

Comparisons of both C3F and C3M levels in the patients in the first cohort (patients with adenocarcinoma and squamous cell carcinoma and the healthy controls) was done performing a Kruskal-Wallis test followed by Dunn's multiple comparisons test. Comparisons of C3F levels in the patients in the second cohort (patients with NSCLC and the healthy controls) and comparisons of C3F levels in the patients in the third cohort (patients with spondyloarthritis and the healthy controls) were done performing a Mann-Whitney test. The diagnostic accuracy of the C3F assay for discriminating healthy subjects from both lung cancer subtypes (adenocarcinoma and squamous cell carcinoma) as well as discriminating patients with the different subtypes from each other, for discriminating healthy subjects from patients with NSCLC, and for discriminating healthy subjects from spondyloarthritis, was assessed with the area under the receiver operating characteristics (AUROC). The same analysis was performed for the C3M measurements to compare with the diagnostic potential of C3F.

Correlation between C3M and C3F measurements was done using Spearman correlation.

Significance was considered with p-values <0.05 as it follows: * 0.01<p<0.05; ** 0.001<p<0.01; *** 0.0001<p<0.001; **** p<0.0001. Statistical analyses were made with GraphPad Prism (version 9.5.0 for Windows or version 10.1.2 for Windows, GraphPad Software, San Diego, California USA, www.graphpad.com).

### Results

### Specificity

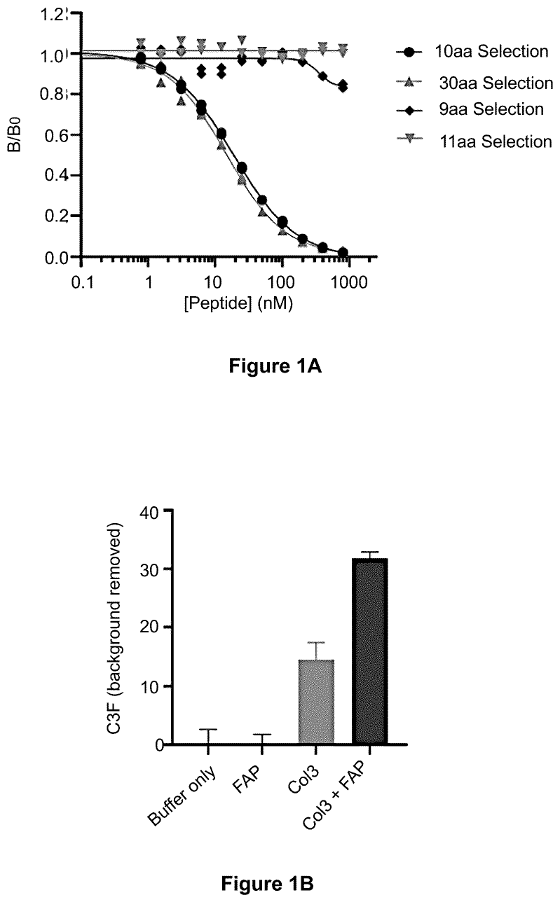

The specificity of the assay was assessed by the ability of different peptides to compete for binding to the monoclonal antibody in a competitive ELISA format. The group of peptides included the 10 and 30 amino acid standard peptides (AGPAGAPGPA (SEQ ID NO: 1) and AGPAGAPGPAGSRGAPGPQGPRGDKGETGE (SEQ ID NO: 19), respectively) corresponding to the N-terminus of the C-terminal fragment generated from FAP cleavage of the alpha-1 chain of type III collagen between amino acid residues P¹⁰⁶⁹ and A¹⁰⁷⁰, an elongated peptide (PAGPAGAPGPA (SEQ ID NO: 2)), and a truncated peptide (GPAGAPGPA (SEQ ID NO: 3)) (FIG. 1A). No cross-reaction was observed for either the elongated or truncated peptide, showing that the C3F assay and antibody recognizes and is specific for the target N-terminus sequence but does not recognize the sequence when located internally within a peptide (and hence lacking the free N-terminus amine group), demonstrating neoepitope specificity of the antibody and assay.

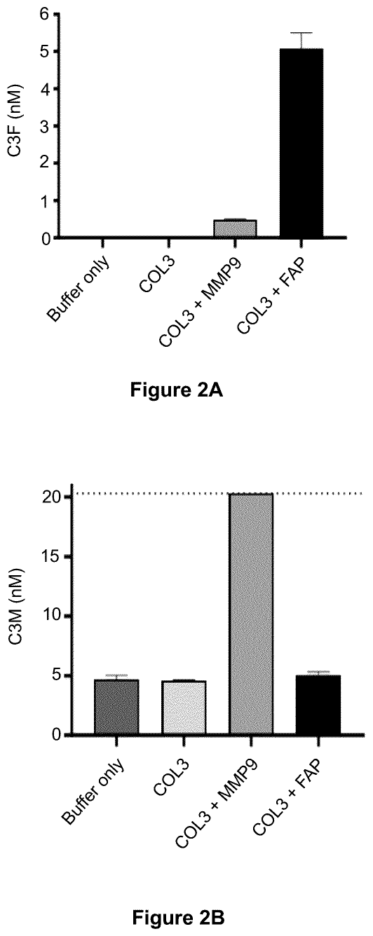

To confirm the assay and antibody's ability to detect FAP cleaved type III collagen and further support the specificity of the ELISA, C3F levels were measured (using the assay) in solutions of recombinant type III collagen after 24 hours of incubation with or without FAP. In initial testing, while a signal was detected from the solution with collagen type III without FAP, there was a clear amplification in signal in the solution with both FAP and type III collagen, indicating that FAP does mediate formation of the fragment from type III collagen and that this fragment can be detected and quantified with the C3F ELISA (FIG. 1B). In further testing, the specificity of the C3F assay for the FAP generated fragment of type III collagen was further confirmed, with no signal being detected from the solution with collagen type III without FAP (FIG. 2A). For comparison, the results of measuring C3M levels (using the C3M assay) in solutions of recombinant type III collagen after 24 hours of incubation with or without MMP9 are also shown (FIG. 2B).

C3F in Serum from Patients with Lung Cancer (First Cohort)

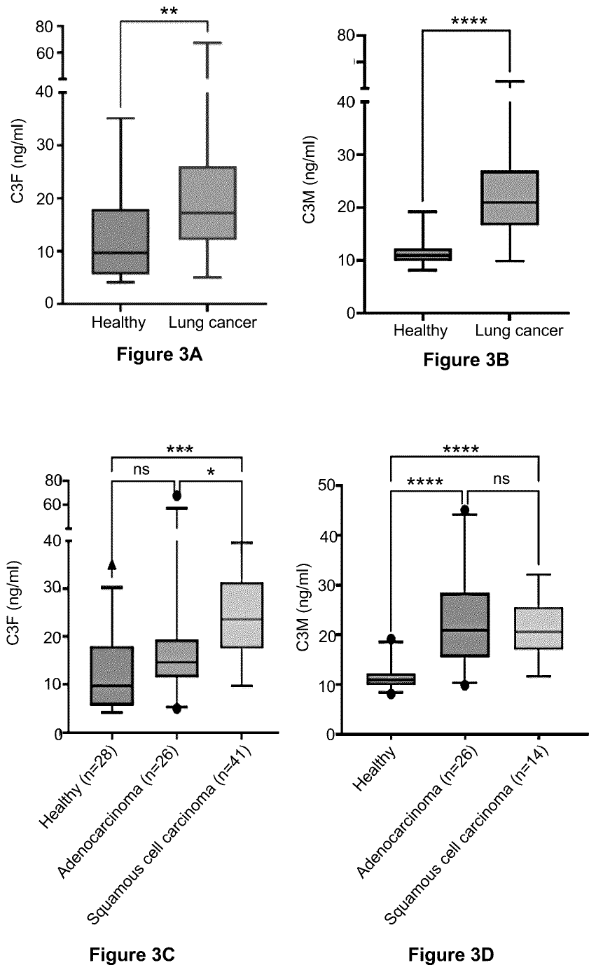

C3F levels were significantly elevated in serum from patients with lung cancer compared to healthy subjects (FIG. 3A), suggesting that the FAP cleaved type III collagen fragment is present in circulation and increased during lung cancer progression. The C3M assay (an established ELISA for measuring MMP-9 cleaved type III collagen) was used to measure C3M levels in the same serum samples, and C3M levels were also shown to be significantly elevated in serum from patients with lung cancer compared to healthy subjects (FIG. 3B).

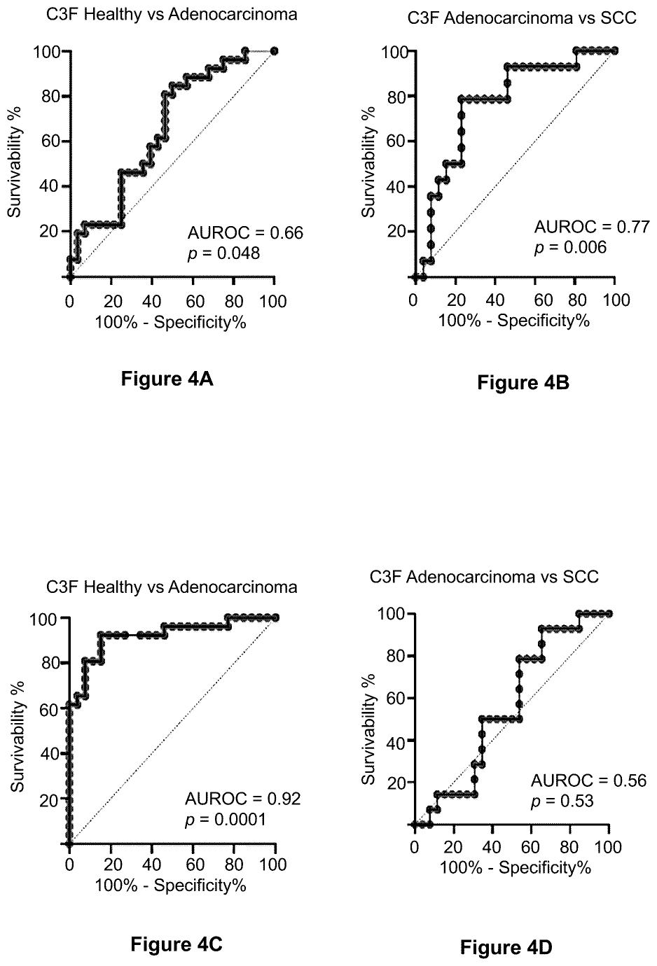

When dividing the patients with lung cancer into patients with adenocarcinoma (n=26) and squamous cell carcinoma (n=14), C3F levels were significantly higher in patients with squamous cell carcinoma compared to patients with adenocarcinoma (FIGS. 3C) and C3F showed better diagnostic accuracy for separating adenocarcinoma from squamous cell carcinoma (AUROC=0.77 p=0.006) (FIG. 4B) than from healthy subjects (AUROC=0.66 p=0.048) (FIG. 4A). In contrast to C3F, C3M could not significantly discriminate patients with adenocarcinoma from patients with squamous cell carcinoma (FIG. 3D). In addition, C3M showed good diagnostic accuracy for separating patients with adenocarcinoma from healthy subjects (AUROC=0.92, p<0.0001) (FIG. 4C) and poor diagnostic accuracy for separating patients with adenocarcinoma from squamous cell carcinoma (AUROC=0.56, p=0.53) (FIG. 4D), which is opposite of what was seen for C3F.

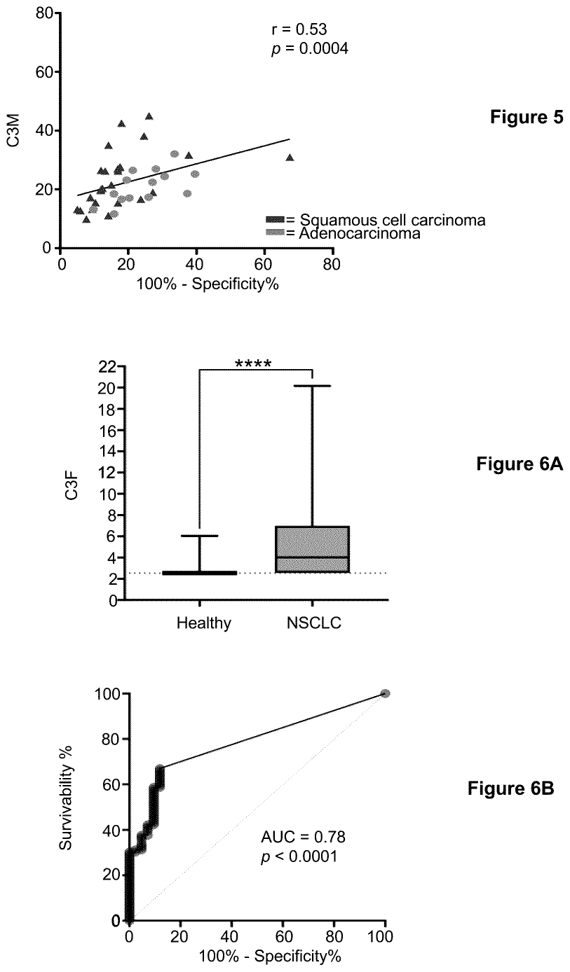

C3F and C3M levels only showed a moderate overall correlation (spearman's r=0.53, p=0.0004) (FIG. 5), further supporting that C3F reflects something different from C3M.

Taken together, these results suggest that the circulating levels of FAP cleaved collagen III fragments (as measured by the C3F assay) reflect different biological processes than circulating levels of MMP-9 cleaved collagen III fragments (as measured by the C3M assay), thus providing additional value and diagnostic potential.

C3F in Serum from Patients with Lung Cancer (Second Cohort)

C3F levels were significantly elevated in serum from patients with NSCLC compared to healthy subjects (p<0.0001) (FIG. 6A) and could significantly discriminate between healthy subjects and patients with NSCLC (AUROC=0.78, p<0.0001) (FIG. 6B), again showing and confirming that the FAP cleaved type III collagen fragment is present in circulation and increased during lung cancer progression. In this cohort, containing a larger number of patients and healthy controls, there was however no difference between C3F levels in patients with adenocarcinoma and patients with squamous cell carcinoma.

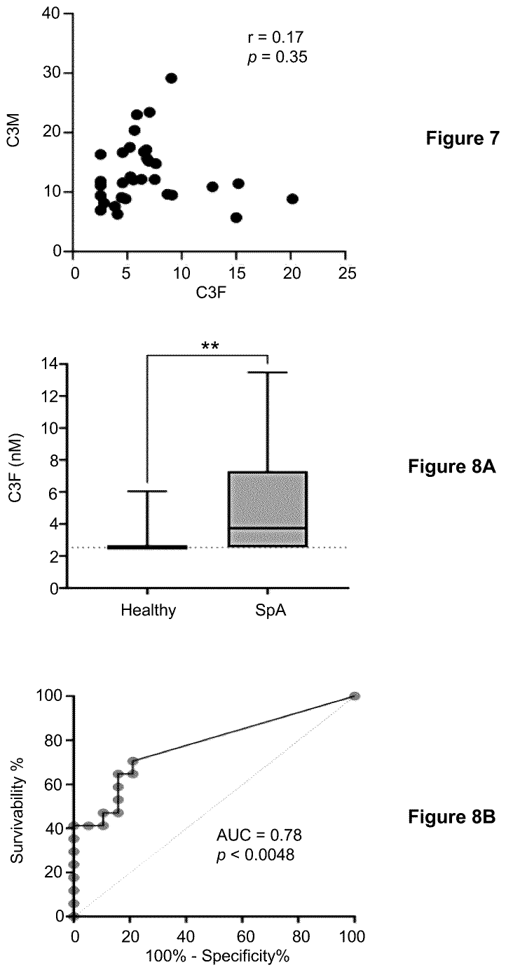

The C3M assay was used to measure C3M levels in a subset of the same serum samples. C3F and C3M levels did not correlate (spearman's r=0.17, p=0.35) (FIG. 7), further supporting that C3F reflects something different from C3M.

Taken together, these results again suggest that the circulating levels of FAP cleaved collagen III fragments (as measured by the C3F assay) reflect different biological processes than circulating levels of MMP-9 cleaved collagen III fragments (as measured by the C3M assay), thus providing additional value and diagnostic potential.

C3F in Serum from Patients with Spondyloarthritis (Third Cohort)

C3F levels were significantly elevated in serum from patients with spondyloarthritis compared to healthy subjects (p<0.0015) (FIG. 8A) and could significantly discriminate between healthy subjects and patients with NSCLC (AUROC=0.78, p<0.0048) (FIG. 8B), suggesting that the FAP cleaved type III collagen fragment in circulation is increased for patients with spondyloarthritis.

### DISCUSSION

The inventors have developed a competitive ELISAs measuring the AGPAGAPGPA (SEQ ID NO: 1)N-terminus sequence, the levels of which reflect FAP activity through FAP cleavage of type III collagen. The immunoassays can detect and quantify the levels of this biomarker in blood-based samples from patients with lung cancer. The biomarker could be distinguished from another type III collagen degradation marker, C3M (MMP-9 mediated cleavage of type III collagen), and showed different diagnostic potential to C3M. The immunoassay methods, kits and monoclonal antibodies disclosed herein can therefore be used to analysing blood-based samples from a patient for detecting, monitoring and/or assessing cancers or arthritis.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used to mean ‘including or consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### REFERENCES

1. Siegel, R. L.; Miller, K. D.; Fuchs, H. E.; Jemal, A. Cancer Statistics, 2022. CA. Cancer J. Clin. 2022, 72, 7-33, doi:10.3322/caac.21708.
2. Zhao, L.; Chen, J.; Pang, Y.; Fu, K.; Shang, Q.; Wu, H.; Sun, L.; Lin, Q.; Chen, H. Fibroblast Activation Protein-Based Theranostics in Cancer Research: A State-of-the-Art Review. Theranostics 2022, 12, 1557-1569, doi:10.7150/thno.69475.
3. Hamson, E. J.; Keane, F. M.; Tholen, S.; Schilling, 0.; Gorrell, M. D. Understanding Fibroblast Activation Protein (FAP): Substrates, Activities, Expression and Targeting for Cancer Therapy. PROTEOMICS—Clin. Appl. 2014, 8, 454-463, doi:10.1002/prca.201300095.
4. Binnewies, M.; Roberts, E. W.; Kersten, K.; Chan, V.; Fearon, D. F.; Merad, M.; Coussens, L. M.; Gabrilovich, D. I.; Ostrand-Rosenberg, S.; Hedrick, C. C.; et al. Understanding the Tumor Immune Microenvironment (TIME) for Effective Therapy. Nat. Med. 2018, 24, 541-550, doi:10.1038/s41591-018-0014-x.
5. Jin, M.-Z.; Jin, W.-L. The Updated Landscape of Tumor Microenvironment and Drug Repurposing. Signal Transduct. Target. Ther. 2020, 5, 166, doi:10.1038/s41392-020-00280-x.
6. Anderson, N. M.; Simon, M. C. The Tumor Microenvironment. Curr. Biol. 2020, 30, R921-R925, doi:10.1016/j.cub.2020.06.081.
7. Desbois, M.; Wang, Y. Cancer-Associated Fibroblasts: Key Players in Shaping the Tumor Immune Microenvironment. Immunol. Rev. 2021, 302, 241-258, doi:10.1111/imr.12982.
8. Barrett, R. L.; Pure, E. Cancer-Associated Fibroblasts and Their Influence on Tumor Immunity and Immunotherapy. Elife 2020, 9, 1-20, doi:10.7554/eLife.57243.
9. Nissen, N. I.; Johansen, A. Z.; Chen, I.; Johansen, J. S.; Pedersen, R. S.; Hansen, C. P.; Karsdal, M. A.; Willumsen, N. Collagen Biomarkers Quantify Fibroblast Activity In Vitro and Predict Survival in Patients with Pancreatic Ductal Adenocarcinoma. Cancers (Basel). 2022, 14, 819, doi:10.3390/cancers14030819.
10. Wu, F.; Yang, J.; Liu, J.; Wang, Y.; Mu, J.; Zeng, Q.; Deng, S.; Zhou, H. Signaling Pathways in Cancer-Associated Fibroblasts and Targeted Therapy for Cancer. Signal Transduct. Target. Ther. 2021, 6, 218, doi:10.1038/s41392-021-00641-0.
11. Alvarez-Teijeiro, S.; Garcia-InclAn, C.; Villaronga, M.; Casado, P.; Hermida-Prado, F.; Granda-Diaz, R.; Rodrigo, J.; Calvo, F.; Del-Rio-Ibisate, N.; Gandarillas, A.; et al. Factors Secreted by Cancer-Associated Fibroblasts That Sustain Cancer Stem Properties in Head and Neck Squamous Carcinoma Cells as Potential Therapeutic Targets. Cancers (Basel). 2018, 10, 334, doi:10.3390/cancers10090334.
12. Fitzgerald, A. A.; Weiner, L. M. The Role of Fibroblast Activation Protein in Health and Malignancy. Cancer Metastasis Rev. 2020, 39, 783-803, doi:10.1007/s10555-020-09909-3.
13. Park, J. E.; Lenter, M. C.; Zimmermann, R. N.; Garin-Chesa, P.; Old, L. J.; Rettig, W. J. Fibroblast Activation Protein, a Dual Specificity Serine Protease Expressed in Reactive Human Tumor Stromal Fibroblasts. J. Biol. Chem. 1999, 274, 36505-36512, doi:10.1074/jbc.274.51.36505.
14. Sdnchez-Garrido, M. A.; Habegger, K. M.; Clemmensen, C.; Holleman, C.; Miller, T. D.; Perez-Tilve, D.; Li, P.; Agrawal, A. S.; Finan, B.; Drucker, D. J.; et al. Fibroblast Activation Protein (FAP) as a Novel Metabolic Target. Mol. Metab. 2016, 5, 1015-1024, doi:10.1016/j.molmet.2016.07.003.
15. Altmann, A.; Haberkorn, U.; Siveke, J. The Latest Developments in Imaging of Fibroblast Activation Protein. J. Nucl. Med. 2021, 62, 160-167, doi:10.2967/jnumed.120.244806.
16. Shi, J.; Hou, Z.; Yan, J.; Qiu, W.; Liang, L.; Meng, M.; Li, L.; Wang, X.; Xie, Y.; Jiang, L.; et al. The Prognostic Significance of Fibroblast Activation Protein-α in Human Lung Adenocarcinoma. Ann. Transl. Med. 2020, 8, 224-224, doi:10.21037/atm.2020.01.82.
17. Lyu, Z.; Li, Y.; Zhu, D.; Wu, S.; Hu, F.; Zhang, Y.; Li, Y.; Hou, T. Fibroblast Activation Protein-Alpha Is a Prognostic Biomarker Associated With Ferroptosis in Stomach Adenocarcinoma. Front. Cell Dev. Biol. 2022, 10, 1-14, doi:10.3389/fcell.2022.859999.
18. Wikberg, M. L.; Edin, S.; Lundberg, I. V.; Van Guelpen, B.; Dahlin, A. M.; Rutegard, J.; Stenling, R.; Oberg, A.; Palmqvist, R. High Intratumoral Expression of Fibroblast Activation Protein (FAP) in Colon Cancer Is Associated with Poorer Patient Prognosis. Tumor Biol. 2013, 34, 1013-1020, doi:10.1007/s13277-012-0638-2.
19. Chen, X.; Song, E. Turning Foes to Friends: Targeting Cancer-Associated Fibroblasts. Nat. Rev. Drug Discov. 2019, 18, 99-115, doi:10.1038/s41573-018-0004-1.
20. Willumsen, N.; Jensen, C.; Green, G.; Nissen, N. I.; Neely, J.; Nelson, D. M.; Pedersen, R. S.; Frederiksen, P.; Chen, I. M.; Boisen, M. K.; et al. Fibrotic Activity Quantified in Serum by Measurements of Type III Collagen Pro-Peptides Can Be Used for Prognosis across Different Solid Tumor Types. Cell. Mol. Life Sci. 2022, 79, 204, doi:10.1007/s00018-022-04226-0.
21. Barascuk, N.; Veidal, S. S.; Larsen, L.; Larsen, D. V.; Larsen, M. R.; Wang, J.; Zheng, Q.; Xing, R.; Cao, Y.; Rasmussen, L. M.; et al. A Novel Assay for Extracellular Matrix Remodeling Associated with Liver Fibrosis: An Enzyme-Linked Immunosorbent Assay (ELISA) for a MMP-9 Proteolytically Revealed Neo-Epitope of Type III Collagen. Clin. Biochem. 2010, 43, 899-904, doi:10.1016/j.clinbiochem.2010.03.012.
22. Zhang, H. E.; Hamson, E. J.; Koczorowska, M. M.; Tholen, S.; Chowdhury, S.; Bailey, C. G.; Lay, A. J.; Twigg, S. M.; Lee, Q.; Roediger, B.; et al. Identification of Novel Natural Substrates of Fibroblast Activation Protein-Alpha by Differential Degradomics and Proteomics. Mol. Cell. Proteomics 2019, 18, 65-85, doi:10.1074/mcp.RA118.001046.

1. A method of immunoassay, said method comprising;
i) contacting a patient sample with a monoclonal antibody that specifically binds to the N-terminus amino acid sequence AGPAGAPGPA (SEQ ID NO: 1); and
ii) detecting and determining the amount of binding between said monoclonal antibody and peptides in the sample.

2. The method as claimed in claim 1, wherein the method is a method of immunoassay for detecting and/or monitoring a cancer or a particular level of severity thereof in a patient, the method further comprising;
iii) correlating said amount of binding with values associated with normal healthy subjects and/or values associated with known disease severity and/or values obtained from said patient at a previous time point and/or with a predetermined cut-off value.

3. The method as claimed in claim 2, wherein the cancer is a lung cancer.

4. The method as claimed in claim 3, wherein the lung cancer is a non-small cell lung cancer.

5. The method as claimed in claim 1, wherein the method is a method of immunoassay for detecting and/or monitoring an arthritis or a particular level of severity thereof in a patient, the method further comprising;
iii) correlating said amount of binding with values associated with normal healthy subjects and/or values associated with known disease severity and/or values obtained from said patient at a previous time point and/or with a predetermined cut-off value.

6. The method as claimed in claim 5, wherein the arthritis is spondyloarthritis.

7. The method as claimed in claim 1, wherein the patient sample is selected from blood, plasma or serum.

8. The as claimed in claim 1, wherein the monoclonal antibody does not specifically bind to a peptide having the N-terminus amino acid sequence PAGPAGAPGPA (SEQ ID NO: 2).

9. The method as claimed in claim 1, wherein the monoclonal antibody does not specifically bind to a peptide having the N-terminus amino acid sequence GPAGAPGPA (SEQ ID NO: 3).

10. The method as claimed in claim 1, wherein the monoclonal antibody is raised against a synthetic peptide having the N-terminus amino acid sequence AGPAGAPGPA (SEQ ID NO: 1).

11. The method as claimed in claim 1, wherein the immunoassay is a competition assay or a sandwich assay.

12. The method as claimed in claim 1, wherein the immunoassay is a radio-immunoassay or an enzyme-linked immunosorbent assay.

13. A monoclonal antibody that specifically binds to the N-terminus amino acid sequence AGPAGAPGPA (SEQ ID NO: 1).

14. The monoclonal antibody as claimed in claim 13, wherein the monoclonal antibody does not specifically bind to a peptide having the N-terminus amino acid sequence PAGPAGAPGPA (SEQ ID NO: 2).

15. The monoclonal antibody as claimed in claim 13, wherein the monoclonal antibody does not specifically bind to a peptide having the N-terminus amino acid sequence GPAGAPGPA (SEQ ID NO: 3).

16. The monoclonal antibody as claimed in claim 13, wherein the monoclonal antibody is raised against a synthetic peptide having the N-terminus amino acid sequence AGPAGAPGPA (SEQ ID NO: 1).

17. An immunoassay kit comprising a monoclonal antibody as claimed in claim 13, and at least one of:
a streptavidin coated well plate;
a biotinylated peptide AGPAGAPGPA-L-Biotin (SEQ ID NO: 14), wherein L is an optional linker;
a secondary antibody for use in a sandwich immunoassay;
a calibrator protein comprising the N-terminus amino acid sequence AGPAGAPGPA (SEQ ID NO: 1);
an antibody biotinylation kit
an antibody HRP labelling kit; or
an antibody radiolabelling kit.


SEQUENCE LISTING (20 sequences)

SEQ ID NO: 1 - protein, 10 aa, synthetic construct
AGPAGAPGPA

SEQ ID NO: 2 - protein, 11 aa, synthetic construct
PAGPAGAPGPA

SEQ ID NO: 3 - protein, 9 aa, synthetic construct
GPAGAPGPA

SEQ ID NO: 4 - protein, 16 aa, Mus musculus
KSSQSLVYSDGKTYMN

SEQ ID NO: 5 - protein, 7 aa, Mus musculus
LVSKLDS

SEQ ID NO: 6 - protein, 9 aa, Mus musculus
WQGTHSPLT

SEQ ID NO: 7 - protein, 5 aa, Mus musculus
SYWMN

SEQ ID NO: 8 - protein, 17 aa, Mus musculus
QIYPGTGDTNYNGKFKG

SEQ ID NO: 9 - protein, 9 aa, Mus musculus
ITTAYYFDY

SEQ ID NO: 10 - protein, 79 aa, Mus musculus
KSSQSLVYSDGKTYMNWLLQRPGQSPKRLVYLVSKLDSGVPDRFTGSGSGTDFTLKISRVEAEDLGVYYCWQGTHSPLT

SEQ ID NO: 11 - protein, 77 aa, Mus musculus
SYWMNWVKQSPGQGLEWIGQIYPGTGDTNYNGKFKGKVTLTTDKSSSTSYMQLSSLTSEDSAVYFCVLITTAYYFDY

SEQ ID NO: 12 - protein, 112 aa, Mus musculus
DVVMTQTPRTLSVIIGQPASISCKSSQSLVYSDGKTYMNWLLQRPGQSPKRLVYLVSKLDSGVPDRFTGSGSGTDFTLKISRVEAEDLGVYYCWQGTHSPLTFGAGTKLELK

SEQ ID NO: 13 - protein, 118 aa, Mus musculus
QVQVQQSGAELVRPGSSVKISCKASGYVFSSYWMNWVKQSPGQGLEWIGQIYPGTGDTNYNGKFKGKVTLTTDKSSSTSYMQLSSLTSEDSAVYFCVLITTAYYFDYWGQGTTLTVSS

SEQ ID NO: 14 - protein, 10 aa, synthetic construct
  MOD_RES 10: note = biotinylated with optional linker
AGPAGAPGPA

SEQ ID NO: 15 - protein, 13 aa, synthetic construct
  MOD_RES 13: note = linked to Keyhole Limpet Hemocyanin
AGPAGAPGPAGGC

SEQ ID NO: 16 - protein, 11 aa, synthetic construct
  MOD_RES 11: note = biotinylated
AGPAGAPGPAK

SEQ ID NO: 17 - protein, 473 aa, Mus musculus
MEWPCIFLFLLSVTEGVHSQVQVQQSGAELVRPGSSVKISCKASGYVFSSYWMNWVKQSPGQGLEWIGQIYPGTGDTNYNGKFKGKVTLTTDKSSSTSYMQLSSLTSEDSAVYFCVLITTAYYFDYWGQGTTLTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 18 - protein, 238 aa, Mus musculus
MSPAQFLLLSVFWIREIKGDVVMTQTPRTLSVIIGQPASISCKSSQSLVYSDGKTYMNWLLQRPGQSPKRLVYLVSKLDSGVPDRFTGSGSGTDFTLKISRVEAEDLGVYYCWQGTHSPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 19 - protein, 30 aa, synthetic construct
AGPAGAPGPAGSRGAPGPQGPRGDKGETGE

SEQ ID NO: 20 - protein, 10 aa, Mus musculus
AGPSGAPGPA